(19) United States
(12) Patent Application Publication
(10) Pub. No.: US 2013/0295131 A1
(43) Pub. Date: Nov. 7, 2013

(54) GENERATION OF ANTIGENIC VIRUS-LIKE PARTICLES THROUGH PROTEIN-PROTEIN LINKAGES

(71) Applicant: Hal S. Padgett, Vacaville, CA (US)

(72) Inventor: Hal S. Padgett, Vacaville, CA (US)

(21) Appl. No.: 13/883,459

(22) PCT Filed: Nov. 5, 2010
(86) PCT No.: PCT/US10/55717

§ 371 Date: May 3, 2013

Related U.S. Application Data
(60) Provisional application No. 61/258,152, filed on Nov. 4, 2009.

(51) Int. Cl. A61K 39/21

(52) U.S. Cl. CPC A61K 39/21
USPC 424/196.11; 530/350

(57) ABSTRACT

We have generated virus-like particles (VLPs) that can display other proteins through covalent protein-protein linkages mediated by the ‘Dock and Lock’ interaction between the Drosophila NorpA protein and the C-terminal pentapeptide tail of the InaD protein. This interaction may also be mediated by a portion of the SITAC protein and the Tetraspanin L6 Antigen protein. This system can be used to generate high-density scaffolded arrays of epitopes for immunization. This technology can streamline VLP vaccine candidate production, making it possible to rapidly evaluate panels of candidates in response to current vaccine needs and emerging pathogen threats.

### CROSS-REFERENCE TO RELATED APPLICATIONS

This application claims priority from U.S. Provisional Application No. 61/258,152, filed Nov. 5, 2009. The prior application is hereby incorporated by reference in its entirety.

### BACKGROUND OF THE INVENTION

Virus like particle (VLP) vaccines are recombinant structures that mimic the overall structure of virus particles and exhibit adjuvant properties capable of inducing neutralizing immune responses. VLPs have been used successfully to protect humans from hepatitis B virus and human papillomavirus infection. A number of VLP platforms have been engineered to display a range of antigens on their surface and are currently being explored for their potential to combat other infectious diseases and cancer.

VLP technology has the potential to allow rapid evaluation of large numbers of candidate antigens, provided such engineered VLP systems are sufficiently adaptable to display the antigens, either alone or in various combinations, with minimal groundwork.

Various VLP platforms are based on viral proteins that can self-assemble without the infectious viral nucleic acid component. Other platforms display heterologous antigenic components directly on the surface of intact virus particles that contain infectious or partially infectious nucleic acid components. Such particles are viral in nature, and are aptly described as modified virus particles, but are also described herein as VLPs because they are typically recombinant and can be used to display heterologous antigens. VLP technologies that involve genetic fusion of antigens to virus coat proteins (CP) and are typically limited to small peptide antigens. Often, the antigens displayed in those systems negatively affect virus particle formation and recovery. This high degree of unpredictability requires that an individualized program of sequence modification, expression analysis, and purification process development must first be carried out for each antigen prior to conducting even preliminary immunological studies.

### BRIEF SUMMARY OF THE INVENTION

Method are described for generating virus-like particles linked to antigens through protein-protein interaction.

In one embodiment, a method of generating a virus-like particle covalently linked to a polypeptide of interest is presented comprising: providing a first polypeptide fused to viral coat protein, and providing a second polypeptide fused to the polypeptide of interest, wherein the first polypeptide and the second polypeptide are capable of protein-protein interaction such that covalent links are formed between the first and second polypeptides via oxidative cross-linking, and wherein the viral coat protein is capable of assembling into a virus-like particle.

In another embodiment, a method of generating a multivalent virus-like particle covalently linked to two or more polypeptides of interest is presented comprising: providing a viral coat protein comprising a Carboxy-terminal fusion with the amino acid sequence TEFCA, and providing two or more different polypeptides of interest individually fused to InaD or a fragment of InaD containing the PDZ1 domain, wherein the TEFCA sequence and the PDZ1 domain are capable of protein-protein interaction such that covalent links are formed via oxidative cross-linking, and wherein the viral coat protein is capable of assembling into a virus-like particle, whereby a multivalent virus-like particle is formed.

In yet another embodiment, a vaccine is described comprising: a first polypeptide fused to viral coat protein, and a second polypeptide fused to an antigen of interest, wherein the first polypeptide and the second polypeptide are capable of protein-protein interaction such that covalent links are formed between the first and second polypeptides via oxidative cross-linking, and wherein the viral coat protein is assembled into a virus-like particle, such that the antigen is displayed on the virus-like particle.

### BRIEF DESCRIPTION OF THE DRAWINGS

FIG. 1 lists the sequence of the U1CP_TEFCA_Direct construct (SEQ ID NO: 06).

FIG. 2 lists the amino acid sequence of the U1CP_TEFCA_Spacer construct (SEQ ID NO: 07).

FIG. 3 lists the amino acid sequence of the polyhistidine-tagged InaD construct (SEQ ID NO: 08).

FIG. 4 lists the amino acid sequence of the polyhistidine-tagged InaD-GFP IGH fusion construct (SEQ ID NO: 09).

FIG. 5 lists the amino acid sequence of the polyhistidine-tagged InaD-GFP GIH fusion construct (SEQ ID NO: 10).

FIG. 6 shows a schematic of ‘Dock & Lock’ intermolecular interactions mediated by GFP fused to the InaD domain and CP fused to the NorpA C-terminal 5 amino acids.

FIG. 7 shows SDS-PAGE gel data demonstrating ‘Dock & Lock’ intermolecular interactions mediated by GFP (green fluorescent protein) fused to the InaD domain and CP fused to the NorpA C-terminal 5 amino acids (left two lanes). Covalent linkage by disulfide bond formation (center lane) was confirmed by treating the joined proteins with reducing agent, which liberated the individual proteins from one-another (right lane).

FIG. 8 is a drawing depicting antigenic VLP production. A modified virus particle serves as a universal structural scaffold for antigen display. The particle can accept various antigens through rapid and specific covalent linkage.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention generally is a method for generating virus-like particles (VLPs) that can display other proteins through covalent protein-protein linkages.

The instant approach helps to overcome many of the shortcomings of traditional VLP production methodologies by separating manufacturing of the VLP scaffold from antigen production. Consequently, testing of new antigens in a VLP format may only require production of the antigenic component.

Large quantities of the universal VLP antigen acceptor scaffold can be produced, eliminating the need for complicated and time-consuming work with recombinant virus constructs. When brought together, the scaffold and recombinant antigens can spontaneously associate and covalently lock together to form mature VLP particles with high-density surface arrays of antigen. For testing new antigens, they can be expressed with a small linkage moiety, and then be mixed with the universal scaffold to form the antigen-specific VLPs.

An important aspect of this approach can be in the mechanism of linkage that is used to associate the coat protein with the antigen protein. Kimple, Siderovski, and Sondek (EMBOJ 20(2001)4414-4422) observed that the N-terminal PDZ domain of InaD could interact with, and covalently bind to, the C-terminal five amino acid sequence of NorpA. Kimple and Sondek went on to describe how that interaction could be used for protein affinity purification and labeling for biochemical detection (BioTechniques 33(2002)578-590; U.S. Pat. No. 7,309,575). However, they did not contemplate the use of this type of linkage for VLP vaccine production.

In another embodiment of the instant invention, other protein pairs may covalently interact in a similar way and may be used to generate VLPs displaying antigens. In the interaction between SITAC and the Tetraspanin L6 Antigen, described by Borrell-Pages, et al., (MolBiolCell 11(2000)4217-4225), covalent interaction is likely but it is less well-characterized than the InaD/NorpA interaction.

In another embodiment of the instant invention it is contemplated that the sequences of the interacting protein domains can be modified to adjust the degree of affinity. Such modifications may be achieved through rational design (for example, see Wedemann et al. J Mol Biol 343(2004)703-718), or through mutation and optimization (see, for example, U.S. patent application Ser. No. 10/637,758, herein incorporated by reference in its entirety).

In another embodiment of the instant invention it is contemplated that any protein capable of forming a VLP, can be decorated with one or more proteins or peptides of interest through the covalent protein-protein interactions described herein.

### EXAMPLE 1

The interaction between the NorpA peptide and InaD was used to mediate covalent interactions between coat protein in intact virus particles and other proteins. For these experiments, the gene sequence encoding the C-terminal pentapeptide (TEFCA, SEQ ID NO: 01) of NorpA was fused to the gene encoding the Tobacco Mosaic Virus (TMV) U1 strain coat protein such that the resultant coat protein (CP) contained a C-terminal extension of the TEFCA (SEQ ID NO: 01) amino acid sequence. The InaD moiety was produced using a TMV-based plant viral vector system in various fusion configurations with the green fluorescent protein (GFP) and/or a poly-histidine tag.

The reducing environment of the cytosol of cells producing either the InaD or NorpA protein fragments can be expected to minimize unwanted crosslinking between the unpaired cysteines of the each protein during expression. Upon lysis, however, the contents of the cells can be released into a potentially oxidative environment, so the presence of anti-oxidants and/or reducing agents during extraction can be useful to facilitate recovery of the protein or virus in an unoxidized and soluble state.

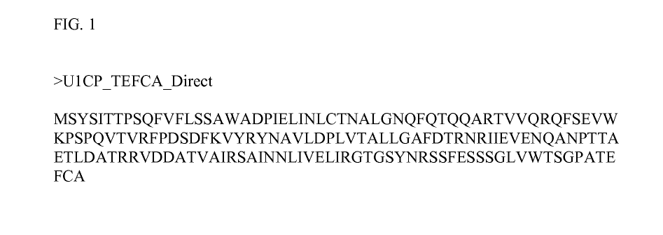

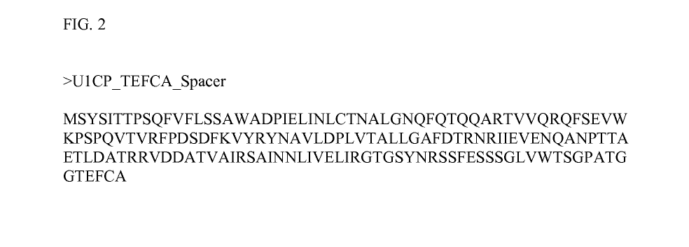

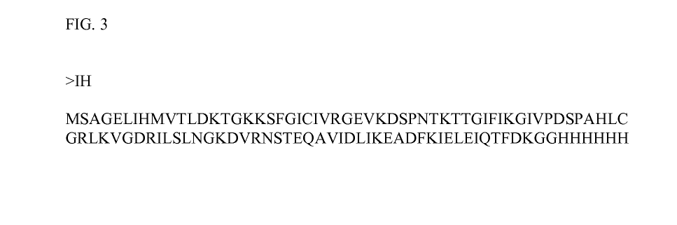

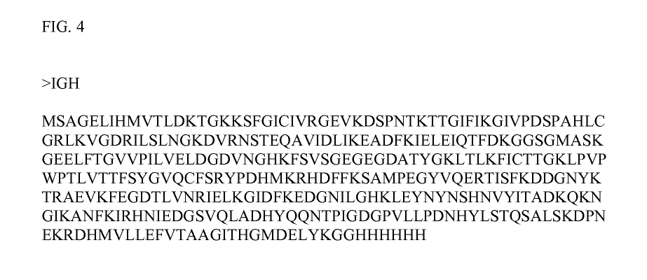

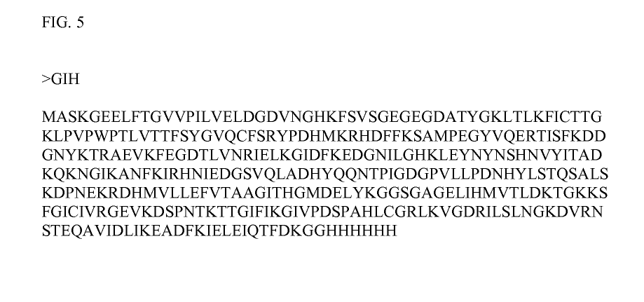

Recombinant virus preparations representing multiple configurations of viral coat protein with a C-terminal TEFCA (SEQ ID NO: 01) sequence were produced. The amino acid sequences of the coat protein-TEFCA fusion for two such preferred constructs, U1CP_TEFCA_Direct and U1CP_TEFCA_Spacer, are shown in FIGS. 1 and 2, respectively. Polyhistidine-tagged InaD, named IH (FIG. 3), and polyhistidine tagged fusions of InaD and the green fluorescent protein, named IGH (FIG. 4) and GIH (FIG. 5), were generated and purified using immobilized nickel affinity chromatography.

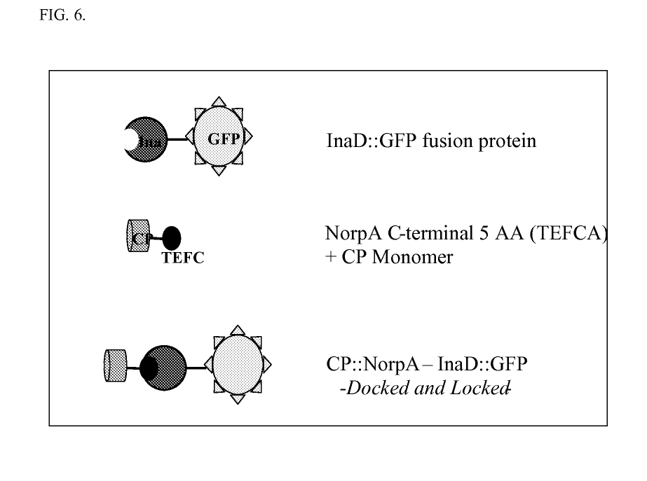

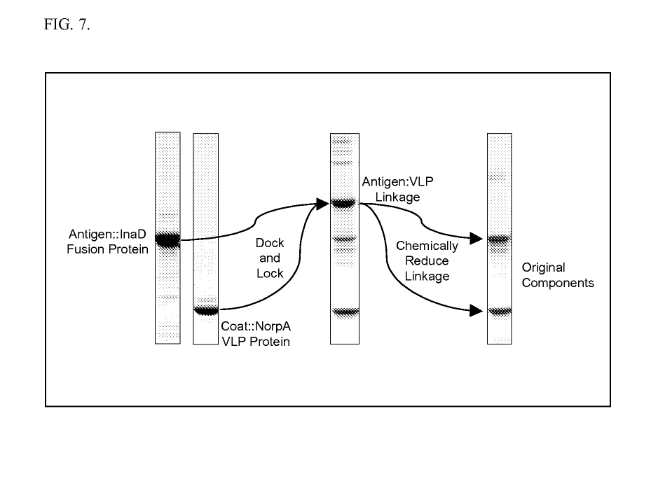

The covalent linkage between U1CP_TEFCA_Spacer and IGH is based on oxidative cross-linking between unpaired cysteines that are brought into juxtaposition by docking of the TEFCA (SEQ ID NO: 01) peptide with InaD domain as diagrammed in FIG. 6. Linkage between the NorpA-modified coat protein and IGH was demonstrated by incubating the virus containing TEFCA-modified coat protein monomers with the various purified InaD fragment-containing proteins. In the example shown in FIG. 7, the two proteins were able to link together to form a species that migrated at the expected position for an entity comprised of the GFP::InaD fusion and the CP-TEFCA fusion. The disulfide nature of the linkage was demonstrated by treatment of the linked protein preparation with beta-mercaptoethanol to reduce the linkage. This treatment eliminated the covalent linkage between the two proteins, allowing each to migrate independently in the gel.

When the polyhistidine-tagged InaD protein was incubated with the TEFCA-modified virus, similar evidence of covalent linkage between the proteins was observed. Moreover, the covalent complexes could be precipitated with 4% polyethylene glycol (MW 8,000) that is used to precipitate viruses, indicating that TEFCA-modified virus particles had been decorated with the InaD protein.

This approach can provide important advantages for VLP technology. Typical approaches to create VLP vaccines often do not accommodate whole proteins, and are based on genetic fusions of antigenic peptides in various positions within the coat protein. Coat proteins with genetic fusions to peptides frequently impair virion assembly or cause other anomalies that can lead to low virion recovery or encouragement of genetic instability leading to loss or change of the sequences encoding the peptide. Other strategies for production of VLPs that display foreign epitopes often require bifunctional chemical cross-linking reagents, or are based on non-covalent interactions between the proteins mediated by avidin:biotin or similar interactions. Those non-covalent interactions, though stable on a timescale of hours to days, may not be sufficiently stable during the time period of days, weeks, or months that may be required for storage prior to their use as immunogens.

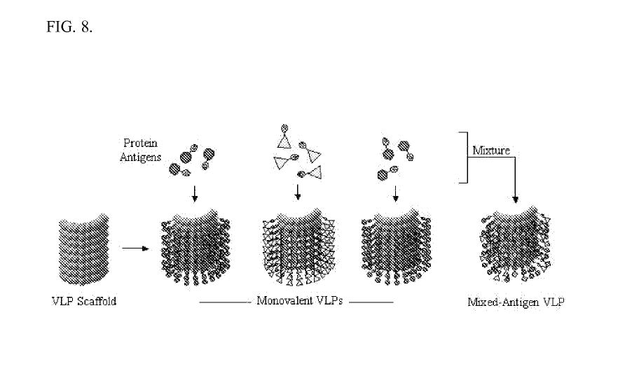

This interaction described herein is specific and covalent and can mediate linkage of the antigen protein to the virus-based VLP scaffold to form VLPs decorated on their surface with high concentrations of antigen. Mixtures of various antigens or other molecules, including immunomodulatory agents such as cytokines or toll-like receptor agonists fused to the InaD domain can also be bound to the VLP scaffold to create multivalent VLP particles (FIG. 8). The ratios between the various antigens can be controlled to obtain particular ratios of each bound to the particle.

In addition to displaying protein antigens on the virus particle surface, the instant system can also be useful for instances where it is desirable to decorate the particle surface with proteins such as enzymes and antibodies for applications in which high-density protein arrays are important, such as for biocatalyst and biosensor applications.

NorpA C-terminal amino acid sequence:

(SEQ ID NO: 02)
...EEEAYKTQGKTEFCA

InaD fragment (13-107):

(SEQ ID NO: 03)
AGELIHMVTLDKTGKKSFGICIVRGEVKDSPNTKTTGIFIKGIVPDSP
AHLCGRLKVGDRILSLNGKDVRNSTEQAVIDLIKEADFKIELEIQTFD
K

Tetraspanin L6 antigen:

(SEQ ID NO: 04)
...GFCCSHQQQYDC

SITAC18:

(SEQ ID NO: 05)
MSSLYPSLED LKVDQAIQAQ VRASPKMPAL PVQATAISPP
PVLYPNLAEL ENYMGLSLSS QEVQESLLQI PEGDSMVAPV
TGYSLGVRRA EIKPGVREIH LCKDERGKTG LRLRKVDQGL
FVQLVQANTP ASLVGLRFGD QLLQIDGRDC AGWSSHKAHQ
VVKKASGDKI VVVVRDRPFQ RTVTMHKDSM GHVGFVIKKG
KIVSLVKGSS AARNGLLTNH YVCEVDGQNV IGLKDKKIME
ILATAGNVVT LTIIPSVIYE HMVKKLPPVL LHHTMDHSIP
DA

It should be understood, of course, that the foregoing relates to exemplary embodiments of the invention and that modifications may be made without departing from the spirit and scope of the invention as set forth in the following claims.

What is claimed is:

1. A method of generating a virus-like particle covalently linked to a polypeptide of interest comprising:
providing a first polypeptide fused to viral coat protein, and
providing a second polypeptide fused to the polypeptide of interest,
wherein the first polypeptide and the second polypeptide are capable of protein-protein interaction such that covalent links are formed between the first and second polypeptides via oxidative cross-linking, and
wherein the viral coat protein is capable of assembling into a virus-like particle.

2. The method of claim 1 wherein the oxidative cross-linking is between unpaired cysteines.

3. The method of claim 1 wherein the protein-protein interaction is between NorpA or a C-terminal fragment of NorpA and InaD or a fragment of InaD containing the PDZ1 domain.

4. The method of claim 1 wherein the viral coat protein is from a plant virus.

5. The method of claim 4 wherein the plant virus is Tobacco Mosaic Virus.

6. The method of claim 1 wherein the polypeptide of interest is an antigen.

7. The method of claim 1 wherein the protein-protein interaction is between portions of SITAC and the Tetraspanin L6 Antigen.

8. The method of claim 1 wherein two or more different polypeptides of interest are attached to the virus-like particle.

9. A method of generating a multivalent virus-like particle covalently linked to two or more polypeptides of interest comprising:
providing a viral coat protein comprising a Carboxy-terminal fusion with the amino acid sequence TEFCA, and
providing two or more different polypeptides of interest individually fused to InaD or a fragment of InaD containing the PDZ1 domain,
wherein the TEFCA sequence and the PDZ1 domain are capable of protein-protein interaction such that covalent links are formed via oxidative cross-linking, and
wherein the viral coat protein is capable of assembling into a virus-like particle,
whereby a multivalent virus-like particle is formed.

10. The method of claim 9 wherein the two or more polypeptides of interest include at least one antigen and at least one immunomodulatory agent.

11. A vaccine comprising:
a first polypeptide fused to viral coat protein, and
a second polypeptide fused to an antigen of interest,
wherein the first polypeptide and the second polypeptide are capable of protein-protein interaction such that covalent links are formed between the first and second polypeptides via oxidative cross-linking, and
wherein the viral coat protein is assembled into a virus-like particle,
such that the antigen is displayed on the virus-like particle.


SEQUENCE LISTING (10 sequences)

SEQ ID NO: 1 - protein, 5 aa, Drosophila melanogaster
TEFCA

SEQ ID NO: 2 - protein, 15 aa, Drosophila melanogaster
EEEAYKTQGKTEFCA

SEQ ID NO: 3 - protein, 97 aa, Drosophila melanogaster
AGELIHMVTLDKTGKKSFGICIVRGEVKDSPNTKTTGIFIKGIVPDSPAHLCGRLKVGDRILSLNGKDVRNSTEQAVIDLIKEADFKIELEIQTFDK

SEQ ID NO: 4 - protein, 12 aa, Homo sapiens
GFCCSHQQQYDC

SEQ ID NO: 5 - protein, 282 aa, Homo sapiens
MSSLYPSLEDLKVDQAIQAQVRASPKMPALPVQATAISPPPVLYPNLAELENYMGLSLSSQEVQESLLQIPEGDSMVAPVTGYSLGVRRAEIKPGVREIHLCKDERGKTGLRLRKVDQGLFVQLVQANTPASLVGLRFGDQLLQIDGRDCAGWSSHKAHQVVKKASGDKIVVVVRDRPFQRTVTMHKDSMGHVGFVIKKGKIVSLVKGSSAARNGLLTNHYVCEVDGQNVIGLKDKKIMEILATAGNVVTLTIIPSVIYEHMVKKLPPVLLHHTMDHSIPDA

SEQ ID NO: 6 - protein, 163 aa, Artificial Sequence
  fusion of TMV U1 CP and TEFCA
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPATEFCA

SEQ ID NO: 7 - protein, 166 aa, Artificial Sequence
  fusion of TMV U1 CP and TEFCA with GG spacer
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPATGGTEFCA

SEQ ID NO: 8 - protein, 107 aa, Artificial Sequence
  polyhistidine-tagged INAD
MSAGELIHMVTLDKTGKKSFGICIVRGEVKDSPNTKTTGIFIKGIVPDSPAHLCGRLKVGDRILSLNGKDVRNSTEQAVIDLIKEADFKIELEIQTFDKGGHHHHHH

SEQ ID NO: 9 - protein, 350 aa, Artificial Sequence
  polyhistidine-tagged INAD-GFP IGH fusion
MSAGELIHMVTLDKTGKKSFGICIVRGEVKDSPNTKTTGIFIKGIVPDSPAHLCGRLKVGDRILSLNGKDVRNSTEQAVIDLIKEADFKIELEIQTFDKGGSGMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKGGHHHHHH

SEQ ID NO: 10 - protein, 348 aa, Artificial Sequence
  polyhistidine-tagged INAD-GFP GIH fusion
MASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKGGSGAGELIHMVTLDKTGKKSFGICIVRGEVKDSPNTKTTGIFIKGIVPDSPAHLCGRLKVGDRILSLNGKDVRNSTEQAVIDLIKEADFKIELEIQTFDKGGHHHHHH